(19) United States
(12) Patent Application Publication
(10) Pub. No.: US 2021/0372650 A1
(43) Pub. Date: Dec. 2, 2021

(54) METHOD AND SYSTEM FOR MONITORING AMBIENT AIR QUALITY

(71) Applicant: Hyde Industries Limited, Lincoln (GB)

(72) Inventor: Stefan Hyde, Lincoln (GB)

(21) Appl. No.: 17/332,156

(22) Filed: May 27, 2021

Related U.S. Application Data
(60) Provisional application No. 63/030,517, filed on May 27, 2020.

(51) Int. Cl. F24F 11/49; F24F 11/39; G16H 10/60

(52) U.S. Cl. CPC F24F 11/49; F24F 2110/50; G16H 10/60; F24F 11/39

(57) ABSTRACT

A method and system is disclosed for determining multiple parameters of air at multiple locations. Based on the determined parameters air quality is determined by comparison of the determined air quality with standard parameters as prescribed by World Health Organization (WHO). Further, a database is maintained for keeping records and trends of the air quality in multiple varied locations at varying times. Based on the maintained air quality parameters the entities are issued health advisory related to their visit to the multiple locations.

### CROSS REFERENCE TO RELATED APPLICATION(S)

This application is related to and claims priority to U.S. Provisional Patent Application No. 63/030,517 filed on May 27, 2020, the contents of which are incorporated by reference herein.

### TECHNICAL FIELD

The present disclosure relates, generally, to ambient air quality monitoring. In particular, it relates to the field of obtaining air quality parameters at various locations and at varying time periods so as to provide related health notifications.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Air pollution is a mix of particles and gases that can reach harmful concentrations both in outside and indoors locations. The effects of air pollution can range from leading to higher disease risks to rising temperatures. A commonly used measure of outdoor air pollution is an Air Quality Index, or AQI which rates the air conditions based on concentrations of pollutants present in the air. There are many kinds of pollutants, and some of the most common and measured air pollutants are: carbon dioxide (CO2), carbon monoxide (CO), sulfur dioxide (SO2), nitric oxide (NO), nitrogen dioxide (NO2), nitrous oxide (N2O), suspended particles (PM 10 or PM 2.5), ozone (O3), soot, smoke, mold, pollen.

Prevalent ambient air quality monitoring techniques require constructing fixed monitoring stations for capturing the air quality of surrounding places. But since construction cost of the monitoring stations is very high, installation of these stations is limited in numbers. Further, the fixed monitoring stations are restricted to being placed at a specific location and do not determine air quality at multiple locations the entity earlier visited and at entity's current location. This leads to the entity's not obtaining enough air quality data at multiple locations that the entity has visited or plans to visit.

In addition to the above, the prevalent air quality monitoring techniques do not facilitate maintaining historical records or trends of the air quality at multiple locations that may impact the entity's health. Also, there is no provision of providing any notification or warning to the entity being affected by the determined deteriorated air quality.

There is, therefore, a need in the art for providing an improved system and method that monitors and maintains the air quality records at different time periods at varying locations and provides a related relevant health care advisory for the entity.

### BRIEF DESCRIPTION OF THE DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

FIG. 1 illustrates network architecture in which or with which an ambient air quality monitoring system can be implemented for determining air quality in accordance with an embodiment of the present disclosure.

FIG. 2 illustrates exemplary functional components of the proposed ambient air quality monitoring system, in accordance with an embodiment of the present disclosure.

FIG. 3 illustrates an exemplary ambient air quality monitor, in accordance with an embodiment of the present disclosure.

FIG. 4A-B illustrates an exemplary ambient air quality monitor being connected to local and global databases, in accordance with an embodiment of the present disclosure.

FIG. 5 is an exemplary computer system in which or with which embodiments of the present invention may be utilized.

### DETAILED DESCRIPTION

The following is a detailed description of embodiments of the disclosure depicted in the accompanying drawings. The embodiments are in such details as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Embodiments of the present invention may be provided as a computer program product, which may include a machine-readable storage medium tangibly embodying thereon instructions, which may be used to program a computer (or other electronic devices) to perform a process. The term “machine-readable storage medium” or “computer-readable storage medium” includes, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

A machine-readable medium may include a non-transitory medium in which data may be stored and that does not include carrier waves and/or transitory electronic signals propagating wirelessly or over wired connections. Examples of a non-transitory medium may include but are not limited to, a magnetic disk or tape, optical storage media such as compact disk (CD) or digital versatile disk (DVD), flash memory, memory or memory devices. A computer-program product may include code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Furthermore, embodiments may be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a machine-readable medium. A processor(s) may perform the necessary tasks.

Systems depicted in some of the figures may be provided in various configurations. In some embodiments, the systems may be configured as a distributed system where one or more components of the system are distributed across one or more networks in a cloud computing system.

Embodiments of the present disclosure relates generally to ambient air quality monitoring. In particular, it relates to the field of obtaining air quality parameters at various locations and at varying time periods so as to provide related health notifications.

In an embodiment, a method and system is provided for determining air quality at indoor and outdoor locations using an ambient quality management system.

In an embodiment, a method and system is provided for informing the entities (herein also referred to as a child, a minor or an infection vulnerable entity) specifically with respect to Parts-per-million (PPM) and Time weighted average (TWA) on all gases.

In an embodiment, a method and system is provided for generating an audible notification that is created on determination of low level air interference. The notification can be generated on a LCD (Liquid Crystal Display) using notification bars. Further, alarm activation is also generated that are specified at low levels as per unit settings.

In an embodiment, the method and system facilitates determination of pollutants such as CO/CO2/NOX that are covered under Polycyclic Aromatic Hydrocarbons (PAHs). The hydrocarbons are organic compounds containing only carbon and hydrogen composed of multiple aromatic rings (organic rings in which the electrons are delocalized).

In an embodiment, the method and system facilitates maintaining a database that records air quality data related to monitored or recorded short and long term exposure data, such as related to Parts-per-million (PPM) and Time weighted average (TWA). Further, the database may store collected air quality data for different geographic locations such as from a car, a campervan, a tent, a home and so forth.

In an embodiment, the method and system facilitates providing an air quality monitoring unit with a corresponding application running on the monitoring unit to determine and notify when the air pollution levels alter or increase over a period of time. The application may facilitate alerting parents/guardians when their kids are within a pollution zone and their health may be getting affected.

In an embodiment, the method and system facilitates maintaining the determined air quality data in a database in a form of a calendar (as per days, months, years) such that the air quality data is available to be reviewed at a later time or back dated data can be made available for issuing health advisory for the entities.

In an embodiment, the method and system facilitates determining commercial vehicles that need to be tracked using GPS to determine the amount of air pollution being caused by these vehicles. The commercial vehicles may be such as a long distance driving trucks, overnights lorries, coach trips and so forth.

In an embodiment, the method and system facilitates the determined air quality data to be saved in the database such that the data can be potentially accrued via the application. The air quality data may be reviewed by WHO (World Health Organization) for looking into health issues of the entity such as related to asthma, learning difficulties and some neurological issues, including birth defects. Further, when the air quality data is correlated via the GPS data the method facilitates determining the air quality data with respect to problem demographics.

Ambient air quality monitoring is a vital part of an ambient air quality monitoring system. Collection of air quality data requires the system to perform steps such as : (a) assessing extent of air pollution, (b) determining air pollution data, (b) providing details of the air pollution data to general public on regular basis, (c) implementation of air quality goals or standards based on air pollution data, (d) evaluating effectiveness of determined emissions control strategies, (e) providing information on air quality trends, and (0 providing data for evaluation of the air quality standards, and (g) support research for determining impact of air pollution on health of general public.

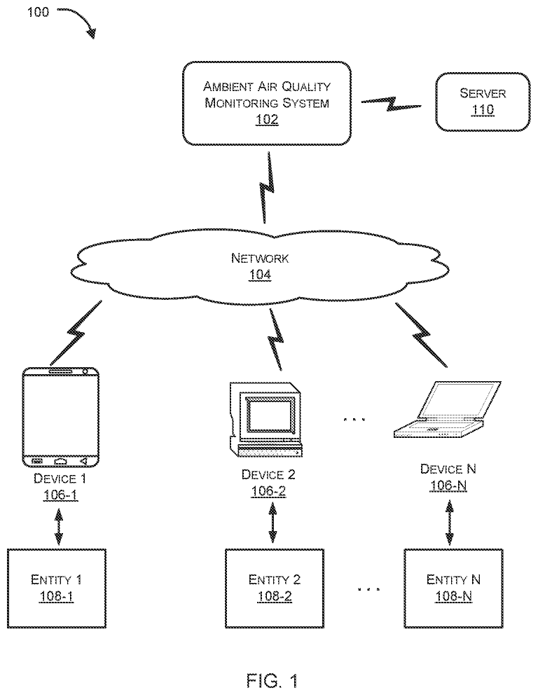

FIG. 1 illustrates a network architecture 100 in which or with which an ambient air quality monitoring system 102 may be implemented for determining outdoor and indoor air quality at a present location of an entity and the various other locations the entity has visited or plans to visit. The determined air quality may be used to determine impact of the air quality on health of the entity in accordance with an embodiment of the present disclosure. In context of the present exemplary architecture 100, the ambient air quality monitoring system 102 (also referred to as the system 102, hereinafter) is described. The ambient air quality monitoring system 102 can be implemented for determining air quality of the air present in surroundings (both indoor and outdoor air) of the entities. The system 102 can be implemented in any computing device and can be configured/operatively connected with a server 110. As illustrated, entities 108-1, 108-2, . . . , 108N (individually referred to as the entity 108 and collectively referred to as the entities 108, hereinafter) can interact with the system 102 using respective entity devices 106-1, 106-2, . . . , 106-N (individually referred to as the entity device 106 and collectively referred to as the entity devices 106, hereinafter), which can be communicatively coupled with the system 102 through a network 104. The entity devices 106 can include a variety of computing systems, including but not limited to, a laptop computer, a desktop computer, a notebook, a workstation, a portable computer, a personal digital assistant, a handheld device and a mobile device.

In an embodiment, the system 102 can be implemented using any or a combination of hardware components and software components such as a cloud, a server, a computing system, a computing device, a network device and the like. In addition, the network 104 can include any number of entity computing devices, such as mobile devices, desktop devices, etc. Illustratively, an entity using the mobile device can interact with an application executing on the mobile device to perform various approval request, response, and notification functions as described herein. Alternatively, or in addition, the entity may interact with a browser-based interface to perform the various approval request, response, and notification functions, such as through a content site (e.g., a “web site” with various “web pages”), a hosted application, or some other interfaces provided by the approval management system and accessed via a browser application executing on the entity device 106.

In an embodiment, the entity 108 can connect to the server 110 via the network 104. The server 110 can be a management server, a web server, or any other electronic device or computing system capable of receiving and sending data. In some embodiments, the server 110 may be a laptop computer, a notebook computer, a tablet computer, a personal computer (PC), a desktop computer, a smartphone, a personal digital assistant (PDA), or any programmable device capable of communication with the entity 108 over the network 104. As can be appreciated by one skilled in the art the entity 108 may be any of a living object, an artificial intelligence and machine learning operationalization software, a machine code, a software program, a deep learning software and so forth. In other embodiments, the server 110 may represent a server computing system utilizing multiple computers as a server system, such as in a cloud computing environment.

In an embodiment, the ambient air quality monitoring system 102 may determine air quality information. The system may determine the air quality data at multiple locations that the entity has earlier visited or is currently located at. Based on the different locations the entity 108 or the entities 108 have visited and for a specific duration, the affect that air pollutants present in the air have over the entities' health is determined and communicated via the network 104. The system 102 may further maintain a record, at a database present at the server 110, of the number of pollutants present in the air and in turn of the level of air pollution. The record stored at the database may be used for later reference and for generating a health advisory.

In yet another embodiment, the system 102 may facilitate mapping the determined level of air quality data present in both indoor and outdoor locations with a geographical map data. The determined level of air quality data may be mapped to the geographical map data. Meteorology data of the locations may be further taken into consideration when combining the determined level of air quality data with the geographical map data. The meteorology data may include elements such as but not limited to wind direction, wind speed, temperature, weather pattern, humidity. In another embodiment, when there is no air quality data available at a specific location, the average of the air quality data of other plural locations closest to the specific location is processed into a result and presented as the air quality data of the location.

In an embodiment, the air quality data mapped to the geographical map data may be used to plan activities for the entities 108 by providing suggestions related to taking precautionary measures for the entities' health. The determined air quality data may be used to determine level of air pollution in real-time. The system 102 may use the air quality data to offer the air pollution free routes and zones to the entities 108.

In an embodiment, the system 102 may facilitate comparing the air quality data with a predefined standard air quality data, e.g., as prescribed by World Health Organization (WHO). Upon the comparison when the air quality data is determined to be in a severe category a warning notification for the entities 108 is generated. The system 102 may generate and issue a warning notification based on different levels of severity of the air quality data. When the abnormality of the air quality data is high a warning notification suggesting the air quality data as abnormal is generated. The warning notification may be sent to the entity device 106 through, for example, a push notification service. The generated warning notification may be for example in any form of visual cues, auditory cues or vibration touch, so as to alert the entity that the air quality is abnormal and not good for heath of the entity.

In an embodiment, the one or more entities can be communicatively coupled with each other through the network 104. Those skilled in the art would appreciate that network 104 can be wireless network, wired network or a combination thereof that can be implemented as one of the different types of networks, such as Intranet, Local Area Network (LAN), Wide Area Network (WAN), Internet, and the like. Further, network 104 can either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like.

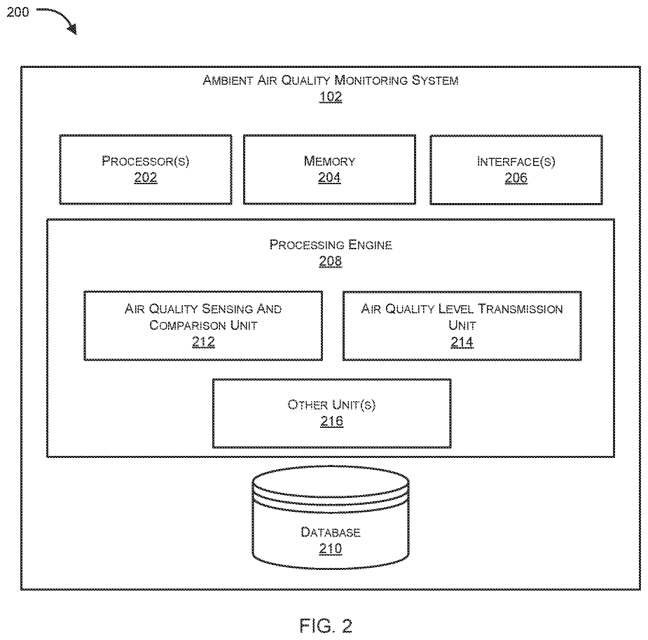

FIG. 2 illustrates exemplary functional components 200 of the proposed ambient air quality monitoring system 102, in accordance with an embodiment of the present disclosure.

In an aspect, the ambient air quality monitoring system 102 may comprise one or more processor(s) 202. The one or more processor(s) 202 may be implemented as one or more microprocessors, microcomputers, microcontrollers, digital signal processors, central processing units, logic circuitries, and/or any devices that manipulate data based on operational instructions. Among other capabilities, the one or more processor(s) 202 are configured to fetch and execute computer-readable instructions stored in a memory 206 of the system 102. The memory 206 may store one or more computer-readable instructions or routines, which may be fetched and executed to create or share the data units over a network service. The memory 206 may comprise any non-transitory storage device including, for example, volatile memory such as RAM, or non-volatile memory such as EPROM, flash memory, and the like.

The system 102 may also comprise an interface(s) 204. The interface(s) 204 may comprise a variety of interfaces, for example, interfaces for data input and output devices, referred to as I/O devices, storage devices, and the like. The interface(s) 204 may facilitate communication of the system 102 with various devices coupled to the system 102. The interface(s) 204 may also provide a communication pathway for one or more components of the system 102. Examples of such components include, but are not limited to, processing engine(s) 208 and database 210.

The processing engine(s) 208 may be implemented as a combination of hardware and programming (for example, programmable instructions) to implement one or more functionalities of the processing engine(s) 208. In examples described herein, such combinations of hardware and programming may be implemented in several different ways. For example, the programming for the processing engine(s) 208 may be processor executable instructions stored on a non-transitory machine-readable storage medium and the hardware for the processing engine(s) 208 may comprise a processing resource (for example, one or more processors), to execute such instructions. In the present examples, the machine-readable storage medium may store instructions that, when executed by the processing resource, implement the processing engine(s) 208. In such examples, the system 102 may comprise the machine-readable storage medium storing the instructions and the processing resource to execute the instructions, or the machine-readable storage medium may be separate but accessible to the system 102 and the processing resource. In other examples, the processing engine(s) 208 may be implemented by electronic circuitry. The database 210 may comprise data that is either stored or generated as a result of functionalities implemented by any of the components of the processing engine(s) 208.

In an exemplary embodiment, the processing engine(s) 208 may comprise an air quality sensing and comparison unit 212, an air quality level transmitting unit 214, and other unit(s) 220.

It would be appreciated that units being described are only exemplary units and any other unit or subunit may be included as part of the system 102 or the processing engine 208. These units too may be merged or divided into super-units or sub-units as may be configured.

In an embodiment, the system 102 facilitates determining quality of ambient air using the air quality sensing and comparison unit 212. The system 102 may determine presence of amount of pollutants, particularly matter, gases and fumes, and vapors in the air. Quantifying type and amount of the pollutants in the air emissions may allow for compliance evaluation of the ambient air with existing or proposed emission regulations. Sampling of the air may be done for determining full-range of regulated pollutants, including criteria pollutants and volatile organic compounds. The pollutants may include components such as metals (including mercury), volatile and semi-volatile organic compounds, acid gases, SO2, NOX, CO, CO2, O2, and so forth.

In an embodiment, the air quality sensing and comparison unit 212 may by using a single sensor or a plurality of sensors (not shown) determine the ambient air quality. The one or more sensors may be such as a temperature sensor, a volatile organic compound sensor (for example, a sensor for sensing formaldehyde and ammonia), a particulate sensor (for example, a PM 2.5 particle sensor), a humidity sensor, a carbon dioxide sensor, an oxygen sensor, a moisture sensor, an ozone sensor, other gas sensors, a carbon monoxide sensor, or a group formed by any combination of the sensors described above, but is not limited thereto. Thus the system 102 may use one or more of the sensors to detect a target such as a volatile organic gas like ammonia or ethanol, or at least one of carbon monoxide, nitrogen dioxide, sulfur dioxide, carbon dioxide, oxygen, a suspended particle, a fine suspended particle, ozone or any combination thereof. The sensor may also sense a virus, a bacterium or a microorganism by a direct or indirect method, and so forth.

In an embodiment, the system 102 through the air quality sensing and comparison unit 212 may determine indoor air quality using the one or more sensors. An indoor air quality (IAQ) monitor may be used to report on multiple levels of common pollutants and other air conditions inside a closed boundary area such as multi-family dwellings, apartment buildings, mobile homes, recreational vehicles, houseboats, motor homes, airplanes, buses, hotels, schools, school rooms, class rooms, sports stadia, concert halls, movie theaters, and the like. Reasons for the indoor pollution may be anything ranging from excessive dust to high humidity to emissions from household cleaners or building materials. For example, one of an indoor pollutant may be a particulate matter (PM) that is a mix of particles and droplets in the air. Multiple indoor activities such as cooking, burning fireplaces, and smoking are a few common sources of generation of the PM. The determined PM as present indoors may be compared with levels of the PM as suggested by the WHO that considers PM 2.5 as an environmental health risk, and suggests that sufficient exposure to the PM 2.5 may lead to irritation of eyes, nose, throat, and lungs, leading to allergy-like symptoms and shortness of breath in otherwise healthy people. Further, higher levels of PM 2.5 may also exacerbate existing medical problems, such as asthma and heart disease. Another type of indoor pollutants is Volatile Organic Compounds (VOCs). Sources of the VOCs are such as hairspray, cosmetics, cleaning fluids, disinfectants, paints, and varnishes.

In an embodiment, the system 102 through the air quality sensing and comparison unit 212 may determine outdoor air quality using the one or more sensors. The outdoor air pollution is a result of factors such as emissions caused by combustion processes from motor vehicles, solid fuel burning and so forth. Other forms of pollution sources of the outdoor pollution include smoke from bushfires, windblown dust, and biogenic emissions from vegetation (pollen and mould spores).

In an embodiment, the system 102 enables tracking air quality data through fixed-point stations, and for variable locations the entity has visited. The air quality data at a given location may enable determining a level of air pollution. Further, the system 102 may comprise means that are associated with the entity 106 for communicating entity location data. The means may be a network-enabled, wearable activity tracker device with a GPS sensor, a Wi-Fi-enabled mobile computer device (e.g., laptop, smartphone, tablet, etc.) where location of the Wi-Fi network's access point may be used to determine the entity's location, and any other suitable location or Global Positioning System (GPS). It may be appreciated by one skilled in the art that an application running on a mobile device, e.g. a smartphone is capable of tracking the movement of the entity using a GPS chip.

In an embodiment, the one or more sensors may be in immediate proximity of the entity, such as on a push chair, a cot, or a buggy. The one or more sensors may be fixed or portable (handheld) or wearable. The wearable sensor may collect air pollution, biometric, activity, and/or location information directly from the entity's body. Further, a stationary sensor may collect air pollution and activity information at a fixed location, such as a home wall or building roof (e.g., NEST indoor sensors, Netatmo sensors and so forth). In yet another embodiment, the sensors may be selectively installed in vehicles such as motorbikes, bicycles, or the entity's portable paraphernalia, such as a helmet worn by a motorbike rider. While the vehicles or the entities keep moving around a vast place the air pollution information of various geographic locations over a vast area is readily obtained.

In an embodiment, the air quality sensing and comparison unit 212 may enable maintaining temporally indexed location information of the entity based on current location, previous visited locations and/or planned locations to be visited of the entity. Determined spatiotemporally indexed air quality data is obtained from the one or more sensors. The one or more sensors may be indexed both in terms of location (e.g., GPS coordinates) and time. The system 102 may compute the air quality data for the entity that are accumulated over a time period (e.g., day, week, month, etc.). The air quality data determined over the time period may be communicated to the entity and its corresponding entities. For example, the entity may determine his/her total air pollution exposure during sleep time in the bedroom, during his/her visit to a park, and during time spent at a workplace. The system 102 may enable the entity to observe that the entity has little air pollution exposure at the workplace, while higher exposure to air pollution, during his/her visit to the park.

In an embodiment, the system by using the air quality transmitting unit 214 may use the sensed and determined air quality data to be stored in the database along with providing the air quality data to the entities using a graphical display device. The graphical display device may be one of a personal computer, a laptop, a smartphone, a table computer, a wearable computer device, etc. The graphical display device may be a display on the entity's computer device or a display on one of the entity's home air quality sensor devices, or any other suitable graphical display device. Further, audio or visual warning indicators may be activated based on the determined air quality and can include alarms, such as horns, buzzers, etc. and flashing light emitting diodes (LEDs). The audio or visual warning indicators and/or dispatched notifications may facilitate informing the entity such as a homeowner, and/or other entities within a residential house, that the air quality has deteriorated or is deteriorating to dangerous levels because of rising levels of air pollutants and/or humidity and/or temperatures. This suggests that relevant and necessary actions needs to be taken to either minimize the amount of air pollutants (e.g., by providing proper ventilation) or by moving to a safe zone with devices such as air conditioners, heaters, coolers, air filters, or air purifiers that may better control air quality.

In an embodiment, as discussed earlier the determined air quality data for multiple locations may be mapped to a geographic map, where various routes and locations on the map may be shown along with the determined air quality for the varied locations. To protect the entities' health the disclosure may facilitate providing precautions against the entities' visit to the polluted area of poor and very poor categories. Further, the entity may be offered a route to for navigation that minimizes his/her visits to the polluted areas.

In an embodiment, the system facilitates providing support of dissemination of air quality results to smart citizens to improve their health. For the same, the system enables creating a real-time conversion service to generate categories of Good, Acceptable, Poor and Very Poor according to comparison of the air quality with the AQI. The proposed system enables helping the entities to be aware of the air quality around them, and by using the air quality data on the map the entities can make informed decisions while planning their activities. The database created for the determined ambient air quality can be used by related entities for creating public awareness of the air pollution around them in real time.

In an embodiment, the system may be used to measure parameters of pollutants as present in the air by such as parts per million (ppm) and time-weighted average (twa) exposure of an adult and raise an alarm when the air is at dangerous levels. For example, the system may determine effect of polluted air on health of an average adult of good lung health when the adult has exposure to polluted air. The system may monitor and record the adults' exposure to the polluted air during a given day/week/month, and based on a consumption of 11,000 liters of air per day would split and take a mean percentage of the polluted air being consumed per day at any given period. Further, this determination of consumption of amount of the polluted air may give the adult more insight (for example, in percentage terms) into how they are treating their lungs and in turn other organs. The system may take into consideration parameters such as adult's BMI, body fat percentage, cholesterol and so forth for determining impact of the polluted air on the adult. Further, by reviewing the determined polluted air exposure data to occurrence of a probable respiratory disease and lung health, the system may form a basis of at what percentage of exposure to the polluted air over time the adult who is exposed to the polluted air is more susceptible to occurrence of respiratory conditions.

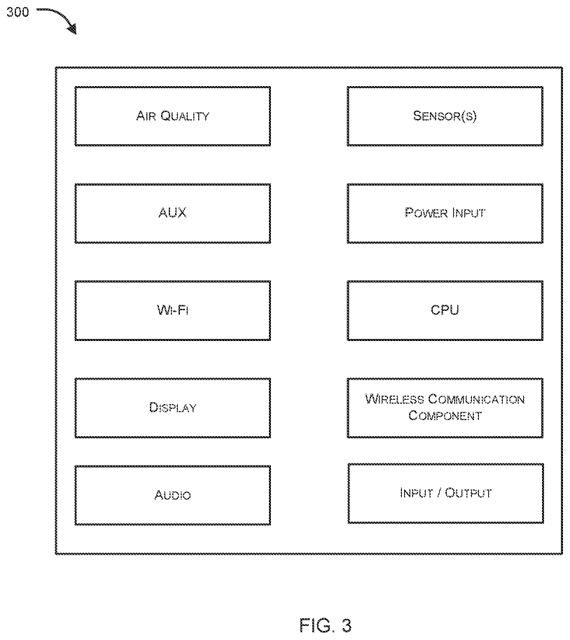

FIG. 3 illustrates an exemplary ambient air quality monitor, 300, in accordance with an embodiment of the present disclosure.

In an embodiment, the air quality data may be collected by the ambient air quality monitor that initially collects an amount of ambient air and determines presence of pollutants within the collected ambient air. The quality of the collected ambient air can be evaluated repeatedly over the TWA on a daily or weekly basis by comparison to set parameters of the air quality standards as determined by the WHO.

In an embodiment, the air quality data present within a residential house or as present in the outdoor air may be monitored such as within boundaries of a residential house or in the outdoor air for determining a deviation from standard air quality parameters as prescribed by the WHO. In response to the deviation from the standard air quality parameters, a notification is transmitted to an air quality monitor to activate an audio/visual warning indicator. As can be appreciated by one skilled in the art the determination of the air quality and thereby determination of the category of the air quality may be determined by operation of a software application or a firmware application executed by one or more processors. The one or more processors may be internal or external to the system and may execute at least a part of the software or firmware application.

Based on the comparison, the application running on the monitor may generate air quality trends and issue warnings via a voicemail about the for example degraded air quality in the surrounding areas. In an embodiment, the application running on the ambient air quality monitor may be personalized for communication with the entities, where the entity can register him/her with the application running on the monitor. The entity can enquire about the air quality in his/her surrounding area by asking the monitor, for example “Hey Buddy, please tell/show current air health?” such that the monitor can issue a warning while referring personally to the entity, such as “hey John, your air readings are good this week”. Further, the monitor can refer to the entity as “Hey John, there appears to be a high reading of (gas) near Grosvenor square on the 19th March”.

In an embodiment, the software application may continuously transfer the captured air quality information to the database maintained on the server. Multiple entities 108 via their associated entity devices 106 (that may be located at disparate locations) may transfer the captured air quality information to the database. The determined air quality data level is compared against a benchmark as established by the WHO. In response to an upward deviation between the air quality data level and the benchmark, the monitor broadcasts a notification to an entity to take remedial action to abate the upward deviation of the air pollution. The deviation may be represented as spikes (e.g., both positive and negative) representing the level of deviation between the determined air quality and the established benchmark. Further, the deviation may be broadcasted by the monitor through use of a short message service (SMS), a multimedia messaging service (MMS), a paging service, an e-mail, or telephonically. Further, in response to an upward deviation between the air quality data level and the benchmark, the server delivers a notification to the air quality monitor to activate an audio/visual warning indicator. The server compares the level of the air pollution against a threshold deviation value that is a function of the benchmark and in response to the level of the air pollution exceeding the threshold deviation, the server broadcasts a notification to the air quality monitor to activate an audio/visual warning indicator. In yet another embodiment, the ambient air quality monitor 300 may include an input/output unit for enabling sensing of the pollutants as present in the air. The determined level of pollutants may be communicated to the entity through the output unit.

In an embodiment, the determined air quality data accumulated in the database present on the server may be categorized into six categories of air quality where the air quality is categorized into parameters such as good with range (0-50), moderate with range (51-100), unhealthy for sensitive groups with range (101-150), unhealthy with range (151-200), very unhealthy with range (201-300) and hazardous with range (301-500). These categories may be increased with increasing effect on human health and may be assigned standard colors for easier identification and reporting. For instance, a red color may be given when the air quality is in the category of very unhealthy and hazardous, a yellow color may be given when the air quality is in the category of unhealthy for sensitive groups and unhealthy, and a green color may be given when the air quality is within the category of good and moderate. The monitor may categorize the determined air quality into one of the relevant categories and display the determined category so as to be used by the entity.

In yet another embodiment, the ambient air quality monitor 300 may facilitate use of fifth generation (5G) technologies for enabling sensing of the pollutants as present in the air on a scale that would be challenging using currently available technologies. The same is done through the input and output unit as shown in FIG. 3. The large amount of pollutant data collected as determined by the input unit may enable air quality sensing and measuring operators to determine levels of air pollution so as to issue a relevant health care advisory to end-consumer services using the output unit. Further, the determined pollutant information may be communicated to a variety of devices for future use. The 5G technology may provide a base for accurate large scale air quality sensing by maintaining energy efficient wireless connections, multiple high precision sensor positioning, sensors connectivity for enabling high bandwidth communications and localized data processing. In an embodiment, the multiple components of the air quality monitor 300 as illustrated in FIG. 3 may function independently, and in combination at a higher speed and with a higher level of efficiency through use of the underlying 5G technologies.

In an embodiment, the entities can upload the determined air quality records as determined by their air quality measuring devices (e.g., one or more sensors) to the air quality monitor. The air quality monitor may further maintain records related to the entities such as a vehicle type used, a determined route, a home size, a heating source, and so forth for determining pollution trends at multiple locations the entity has visited and various sources of the same. This may be used to instruct the entities to avoid the determined sources and locations so as to improve their health conditions.

In an embodiment, one or more base settings of the air quality monitor can be done by the associated entity. These settings can be done through a voice command, where the entities can be determined based on varying lung conditions of the entities, such as (a) a child under certain age, (b) a healthy adult, and (c) an elderly with poor lung health. The settings can be configured using the voice command, where the entity can instruct the monitor to select a setting such that an interface of the monitor may change the settings and reply back as the required settings being done.

In an embodiment, the exemplary air quality monitor unit has a controller that switches a cluster of relays based on threshold values set in software relating to the air quality of a predetermined area (e.g., indoor area or outdoor area). The air quality monitor may communicate, preferably wirelessly, with or one or more sensors. The air quality monitor may be designed to monitor and communicate alert levels for key functions related to air quality within the predetermined area. Communication of the air quality data may be provided to the entities through a cloud based network, or directly to an entity, through, for example, a cell phone using SMS text or other communication mechanisms. The air quality monitor may contain a wireless communication protocol which allows for wireless communication with nearby devices, phones, interne, for uploading information to a cloud based network, checking for update requests, and downloading new software updates. In addition to WI-FI, other wireless communication protocols such as Bluetooth, Zigbee, and SoW, may be used. The air quality monitor may contain a plurality of displays where the displays may be in a form of colored lights (LEDs) which indicate functionality, where green means the air quality being within the determined air quality benchmark, and red means the air quality being within out of the determined air quality benchmark. Further, the air quality monitor has a USB rechargeable or docketing station.

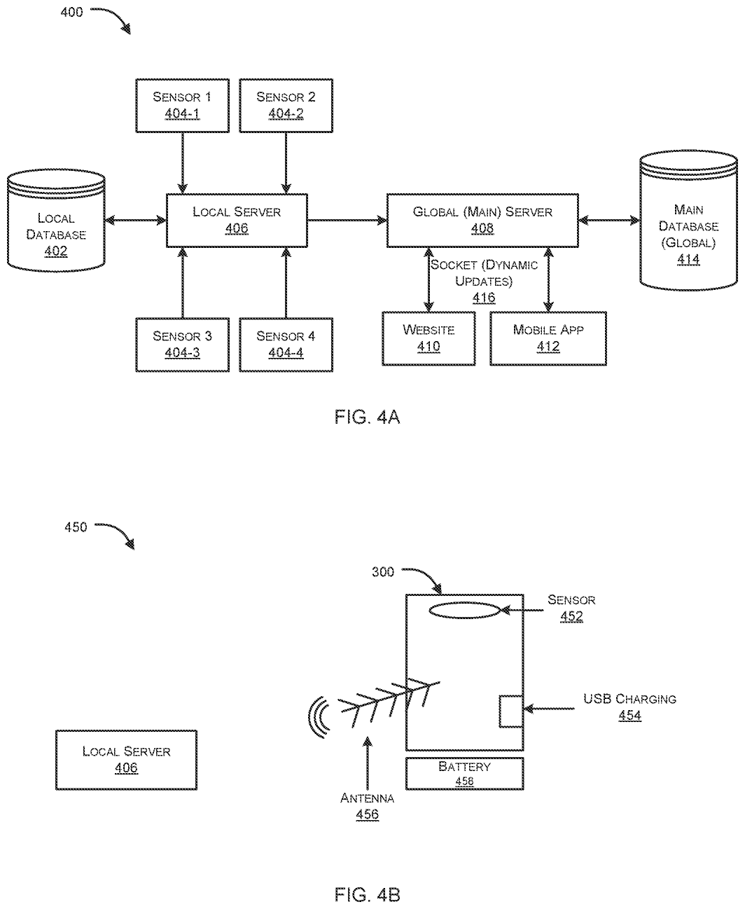

FIG. 4A-B illustrates an exemplary ambient air quality monitor being connected to local and global databases, in accordance with an embodiment of the present disclosure.

In an exemplary implementation, at 400 the sensors 404-1, 404-2 . . . 404-4 of the ambient air quality monitor may be connected to a local server 406 for providing the captured air quality measurements. The local server 406 may be connected to a local database 402 to pass on the information as captured from the sensors. Also, the local server 406 may transfer the information to a global (main) server 408. Further, information from the sensors as maintained at the global (main) server 408 may be accessed via a website 410 and a mobile app 412 that connects with the global server 408 via a socket 416. The website 410 and the mobile app 412 may receive dynamic updates via the socket 416. In another implementation, the global server 408 may be connected to a main database (global) 414 for storing and saving the received information for future reference and use.

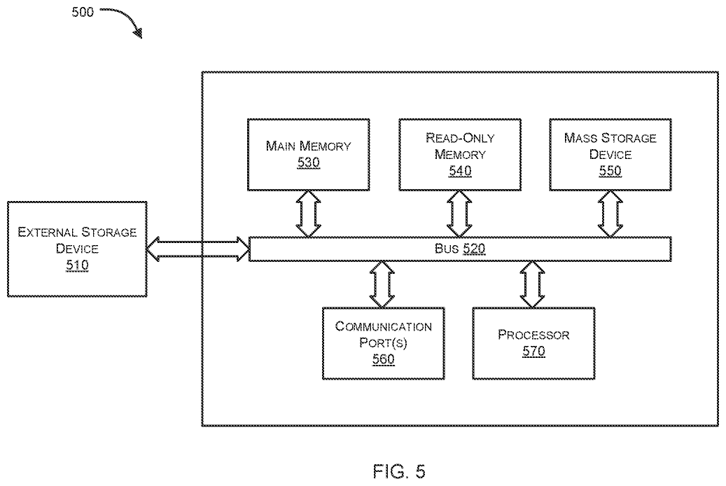

In an exemplary implementation, at 450, a local server 406 (e.g., using raspberry communication mechanism) may wirelessly be connected to the ambient air quality monitor 300. As shown the ambient air quality monitor 300 may have one or more sensors 452 for capturing the air quality and a USB charging slot 454 for receiving power from a power source. In addition, the ambient air quality monitor 300 may have a battery 458 where the power can be stored and used in case of no power supply from a direct power source. Also, the ambient air quality monitor 300 may be equipped with an antenna for receiving and sending information/instructions from a e.g., remotely located local server 406. FIG. 5 is an exemplary computer system, 500 in which or with which embodiments of the present invention may be utilized.

As shown in FIG. 5, computer system includes an external storage device 510, a bus 520, a main memory 530, a read only memory 540, a mass storage device 550, a communication port 560, and a processor 570. Computer system may represent some portion of the approval management system 102.

Those skilled in the art will appreciate that computer system 500 may include more than one processor 570 and communication ports 560. Examples of processor 570 include, but are not limited to, an Intel® Itanium® or Itanium 2 processor(s), or AMD® Opteron® or Athlon MP® processor(s), Motorola® lines of processors, FortiSOC™ system on a chip processors or other future processors. Processor 570 may include various modules associated with embodiments of the present invention.

Communication port 560 can be any of an RS-232 port for use with a modem based dialup connection, a 10/100 Ethernet port, a Gigabit or 10 Gigabit port using copper or fiber, a serial port, a parallel port, or other existing or future ports. Communication port 560 may be chosen depending on a network, such a Local Area Network (LAN), Wide Area Network (WAN), or any network to which computer system connects.

Memory 530 can be Random Access Memory (RAM), or any other dynamic storage device commonly known in the art. Read only memory 540 can be any static storage device(s) e.g., but not limited to, a Programmable Read Only Memory (PROM) chips for storing static information e.g. start-up or BIOS instructions for processor 570.

Mass storage 550 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

Bus 520 communicatively couples processor(s) 570 with the other memory, storage and communication blocks. Bus 520 can be, e.g. a Peripheral Component Interconnect (PCI)/PCI Extended (PCI-X) bus, Small Computer System Interface (SCSI), USB or the like, for connecting expansion cards, drives and other subsystems as well as other buses, such a front side bus (FSB), which connects processor 570 to software system.

Optionally, operator and administrative interfaces, e.g. a display, keyboard, and a cursor control device, may also be coupled to bus 520 to support direct operator interaction with computer system. Other operator and administrative interfaces can be provided through network connections connected through communication port 560. External storage device 510 can be any kind of external hard-drives, floppy drives, IOMEGA® Zip Drives, Compact Disc-Read Only Memory (CD-ROM), Compact Disk-Re-Writable (CD-RW), Digital Video Disk-Read Only Memory (DVD-ROM). Components described above are meant only to exemplify various possibilities. In no way should the aforementioned exemplary computer system limit the scope of the present disclosure.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

What is claimed is:

1. A system for reducing impact of air pollution for an entity, the system comprising:
one or more sensors configured to detect one or more pollutants in ambient air at one or more locations associated with the entity;
a processing unit operatively coupled to a memory, the memory storing instructions executable by the processing unit for configuring the processing unit to:
determine, based at least in part on said detecting, a quality of the ambient air at the one or more locations;
ascertain, based at least in part on said determining, a health profile associated with the entity, and an entity profile, pollution exposure for the entity at the one or more locations over a period of time;
generate, based at least in part on said ascertaining, personalized health advisory for the entity; and
send, over a network, one or more data packets including information related to one or more of the quality of the ambient air, the pollution exposure for the entity, and the personalized health advisory to a device associated with the entity.

2. The system of claim 1, wherein said determining the quality of the ambient air is based on evaluation of one or more parameter values associated with at least a pollutant in the one or more pollutants against a set of standard values retrieved from a database.

3. The system of claim 2, wherein the processing unit is configured to store into and retrieve from the database, one or more of the quality of the ambient air, the one or more parameter values associated with at least a pollutant in the one or more pollutants, the health profile associated with the entity, the entity profile, the ascertained pollution exposure for the entity, and the generated personalized health advisory for the entity.

4. The system of claim 2, wherein said the processing unit is configured to transmit an alert to the device based on determining that the quality of the ambient air fails to satisfy a first threshold, or a parameter value associated with at least a pollutant in the one or more pollutants fails to satisfy a second threshold, or the pollution exposure for the entity fails to satisfy a third threshold.

5. The system of claim 2, wherein the processing unit is configured to determine one or more trends based on evaluation of one or more parameter values associated with at least a pollutant in the one or more pollutants.

6. The system of claim 1, wherein the entity profile includes at least a parameter associated with the entity and at least a preference of the entity.

7. The system of claim 1, wherein the processing unit is configured to compute a route from a source location to a destination location based on any or a combination of the quality of the ambient air, the pollution exposure for the entity and the health profile associated with the entity.

8. The system of claim 1, wherein the processing unit is configured to calculate an average of the quality of the ambient air for a specific location different from the one or more locations based on the determined quality of the ambient air at a plurality of locations of the one or more locations such that the plurality of locations are closest to the specific location.

9. The system of claim 1, wherein the one or more locations include indoor location and outdoor location.

10. The system of claim 1, wherein the information related to one or more of the quality of the ambient air and the pollution exposure for the entity is transmitted to a device associated with a different entity.

11. A method for reducing impact of air pollution for an entity, the method comprising:
detecting, by one or more sensors associated with a computing device, one or more pollutants in ambient air at one or more locations associated with the entity;
determining, by a processing unit of the computing device, based at least in part on said detecting, a quality of the ambient air at the one or more locations;
ascertaining, by a processing unit of the computing device, based at least in part on said determining, a health profile associated with the entity, and an entity profile, pollution exposure for the entity at the one or more locations over a period of time;
generating, by a processing unit of the computing device, based at least in part on said ascertaining, personalized health advisory for the entity; and
sending, by a processing unit of the computing device, over a network, one or more data packets including information related to one or more of the quality of the ambient air, the pollution exposure for the entity, and the personalized health advisory to a device associated with the entity.

12. The method of claim 11, wherein said determining the quality of the ambient air is based on evaluation of one or more parameter values associated with at least a pollutant in the one or more pollutants against a set of standard values retrieved from a database.

13. The method of claim 12, further comprises: storing, in the database, one or more of the quality of the ambient air, the one or more parameter values associated with at least a pollutant in the one or more pollutants, and the ascertained pollution exposure for the entity.

14. The method of claim 12, wherein an alert is transmitted to the device if the determined quality of the ambient air fails to satisfy a first threshold or a parameter value associated with at least a pollutant in the one or more pollutants fails to satisfy a second threshold.

15. The method of claim 12 further comprising: determining one or more trends based on evaluation of one or more parameter values associated with at least a pollutant in the one or more pollutants.

16. The method of claim 11, wherein the entity profile includes at least a parameter associated with the entity and at least a preference of the entity.

17. The method of claim 11 further comprising: computing a route from a source location to a destination location based on any or a combination of the quality of the ambient air, the pollution exposure for the entity and the health profile associated with the entity.

18. The method of claim 11 further comprising: calculating an average of the quality of the ambient air for a specific location different from the one or more locations based on the determined quality of the ambient air at a plurality of locations of the one or more locations such that the plurality of locations are closest to the specific location.

19. The method of claim 11, wherein the one or more locations include indoor location and outdoor location.

20. The method of claim 11, wherein the information related to one or more of the quality of the ambient air and the pollution exposure for the entity is transmitted to a device associated with a different entity.

21. A non-transitory computer-readable storage medium embodying a set of instructions, which when executed by one or more processors of a computer system, causes the one or more processors to perform a method comprising:
detecting, by one or more sensors associated with a computing device, one or more pollutants in ambient air at one or more locations associated with the entity;
determining, by a processing unit of the computing device, based at least in part on said detecting, a quality of the ambient air at the one or more locations;
ascertaining, by a processing unit of the computing device, based at least in part on said determining, a health profile associated with the entity, and an entity profile, pollution exposure for the entity at the one or more locations over a period of time;
generating, by a processing unit of the computing device, based at least in part on said ascertaining, personalized health advisory for the entity; and
sending, by a processing unit of the computing device, over a network, one or more data packets including information related to one or more of the quality of the ambient air, the pollution exposure for the entity, and the personalized health advisory to a device associated with the entity.
